(19) United States
(12) Patent Application Publication
(10) Pub. No.: US 2024/0196945 A1
(43) Pub. Date: Jun. 20, 2024

(54) FERMENTATION AND DEHYDRATION PROCESSES FOR COMMERCIAL PRODUCTION OF FERMENTED NUTS AND SEEDS

(71) Applicants: Timothy Ryan Richards, Stanta Cruz, CA (US); Steven Shane Peterson, Soquel, CA (US)

(72) Inventors: Timothy Ryan Richards, Stanta Cruz, CA (US); Steven Shane Peterson, Soquel, CA (US)

(21) Appl. No.: 18/543,997

(22) Filed: Dec. 18, 2023

Related U.S. Application Data
(60) Provisional application No. 63/433,121, filed on Dec. 16, 2022.

(51) Int. Cl. A23L 25/00; A23B 7/022; A23B 7/155; A23B 9/08; A23B 9/28; B65B 1/00; B65B 3/00; B65B 5/06; B65B 55/12; C12N 1/20

(52) U.S. Cl. CPC A23L 25/40; A23B 7/022; A23B 7/155; A23B 9/08; A23B 9/28; A23L 25/20; B65B 1/00; B65B 3/00; B65B 5/06; B65B 55/12; C12N 1/20

(57) ABSTRACT

A method is provided for fermenting and dehydrating food items such as nuts and seeds to create novel postbiotic foods with numerous benefits. The method comprises preparing a brine solution and adding it with the food item, a sugar product, and select live bacteria cultures to a fermentation vessel. The vessel ferments for 10-14 days at 60-64° F. until a pH of 3.3-4.0 is achieved. The food item is strained, placed on dehydrator trays, and dried at two temperatures to reduce moisture. The process increases nutrition, digestibility, gut health, and safety by reducing antinutrients, agricultural pollutants, toxins, and pathogens by over 5 logs, exceeding pasteurization requirements. The dehydrated food can be packaged or further processed into items like nut butters. The method generates durable, functional nutrition without damaging heat or chemicals.

### FIELD OF THE INVENTION

This invention relates to food processing, and more particularly to a method of fermenting and dehydrating novel postbiotic foods, such as nuts, seeds, legumes, and the like, as well as products derived from these foods, including snack nuts, nut butters, protein powders, nut flours, encapsulated postbiotic supplements, probiotic brines, and the like.

### BACKGROUND

Pathogenic outbreaks and recalls associated with contaminated dry foods have brought the attention of the scientific community and industry to the increasingly important public concerns about these commodities. Salmonellosis outbreaks in the United States and Canada during 2000 to 2004 and in Sweden during 2005 to 2006 involving Salmonella enterica serovar Enteritidis PT 30 were associated with consumption of whole raw almonds and some outbreaks occurred thereafter.

Since current sanitation practices cannot sufficiently ensure the safety of almonds, different pasteurization processing methods have been proposed, ranging from nonthermal, such as high hydrostatic pressure and propylene oxide fumigation, to thermal, including infrared, hot water/air, steam, and radio frequency heating. Thermal processes are preferred by the food industry since they do not leave chemical residues and are relatively easy to perform.

The Almond Board responded by approaching the USDA and requesting a pasteurization mandate, which was passed into law in 2007, requiring the pasteurization of all almonds grown in California. Since then, many health food companies and consumers have resented this mandate, and have gotten around it by importing unpasteurized almonds from Spain/Italy/Turkey, all of which are exempt from the ruling.

Thus, there is a need for a sterilization process consistent with the Almond Board of California's Technical Expert Review Panel's (TERP's) accepted proprietary processes for sterilization (a minimum 5-log reduction) or mandatory treatment (a minimum 4-log reduction) of natural almonds, while maximally retaining the nutritional benefits of almonds.

There are currently two proprietary processes that have been reviewed and accepted by TERP. These processes include specific sets of parameters for the treatment of almond kernels. The two accepted processes are: FMC JSP-1 pasteurization system installed at Going Nuts (Madera, California) and H20 Express pasteurization system Chamber 1, installed at the Stewart & Jasper Company, (Newman, California).

Both proprietary processes utilize steam to accomplish the desired log reduction. Each process has undergone extensive validation testing using almonds inoculated with Salmonella Enteritidis PT 30 (SEPT 30). Each process has established critical control points for one or two sets of operating parameters that have been accepted by TERP (see https://www.almonds.com/sites/default/files/2020-05/proprietary-processes.pdf).

However, both processes involve heating the almonds above 200 degrees F., which can denature proteins, destroy enzymes, generate acrylamide, and render these seeds unviable. Moreover, such processes add an additional expensive and burdensome processing step to farmers and an added expense for farmers and processors, and earn the ire of consumers who want to eat truly unpasteurized almonds straight from the tree.

Propylene oxide (PPO) is a chemical type of pasteurization that uses a known carcinogenic compound to sterilize food meant for human consumption, which is undesirable.

Radio frequency (RF) sterilization is the most-nearly non-heat or chemical pasteurization alternative, but it still heats the almonds to some degree and arguably denatures them in its own way. Many consumers are actively seeking almonds that have not been irradiated (see https://www.cornucopia.org/2016/07/step-towards-truly-raw-almonds/).

Therefore, there is a need for a method of sterilizing almonds and other nuts, seeds, and legumes without denaturing them.

Furthermore, one-third of American households have reported at least one member with food allergies, intolerances, or sensitivities. (International Food Information Council Foundation 2019 Food & Health Survey.) Because two of the top 9 allergens are tree nuts and peanuts, there is a need to employ processes that can reduce allergenicity of these important plant-based proteins.

### SUMMARY

The present invention is a method of fermenting and dehydrating a food item to achieve numerous functional benefits including those laid out in the background above.

Once all equipment and food contact surfaces used in the method are cleaned and sanitized, a 3% brine solution is prepared that consists of a salt product and water. Preferably the salt product is a natural, unrefined ancient seabed salt product containing minerals, such as marketed under the brand Redmond Real Salt. The water is preferably purified spring water.

The water and salt are then mixed until the salt is fully dissolved, at which point the brine should be tested using a salometer, in order to ensure a 3% solution is achieved. Next, a sugar syrup product is added to the brine solution. Such a sugar syrup product is preferably date syrup at a concentration of preferably substantially 1.0% of the total recipe. In our experiments detailed in footnotes, we found that date syrup was the most effective sugar source for enhancing the fermentation and lowering the pH to a level below 4.0 by day 10, as it also contains minerals to support fermentation.

Either hand mix the brine using a long stainless steel mixing paddle, or attach an electric paddle mixer to the brine barrel/tank lid through the bunghole, place the paddle mixer into the brine barrel/tank, and run the paddle mixer until the salt is fully dissolved.

While the brine is mixing, the food item is added to a fermentation vessel, such as the ORBIS® MultiTanks we detail in our recipes in footnotes, along with a mixed culture of lactic acid bacteria (hereto referred to as LAB), which contains heterofermentative and homofermentative strains in a concentration of substantially 0.08%. The LAB starter culture is a mixed LAB culture containing the following strains: Lactobacillus plantarum, Leuconostoc mesenteroides, and Pediococcus acidilactici.

Once the brine is properly mixed, the salt and date syrup are fully dissolved, and the salinity is confirmed, transfer the brine into the fermentation vessels at the specified quantity. This can be accomplished by using a brine pump, by using an elevated brine tank with a tap on it to gravity feed the brine, or if using buckets this can be done by hand.

Constant agitation of the brine is required throughout the filling process in order to ensure that the mineral particles are in solution and equally distributed between the vessels. If the brine is not agitated properly then some vessels will likely contain more minerals than others, which will have an effect on the fermentation process, and ultimately lead to inconsistency in the quality and sensory profiles of the finished product from batch to batch. In order to get the mineral solids into the fermentation vessels, it helps to agitate and continue mixing the brine solution as you make the transfer to the fermentation vessel.

The food item should comprise 40-44% of the recipe as solids, and the remainder is the brine (salt, culture, and date syrup dissolved in water)—56-60% liquids (exact percentages depend on the size and absorptivity of the food item).

Lightly mix the ingredients in the fermentation vessel using a long paddle mixer in order to equally distribute the ingredients, especially the culture, throughout the fermentation medium.

The fermentation vessel is then sealed with an airlocked fermentation system in such a manner that it can offgas CO2 while also facilitating an anaerobic environment inside the vessel. Immediately move the properly sealed vessel into the climate controlled fermentorium and set for between ten to fourteen days in an environment at a temperature of substantially 62-degrees F. (but ranging from 60 to 64 degrees F.).

Between seven and fourteen days the pH of the fermentation solution is checked to see if it is within a specified target acidity pH range of between 3.3 pH and 4.0 pH. Once the fermentation solution has achieved a pH of 4.0 or lower, the food item is strained from the brine solution and optionally spun to remove any liquid from the food item, which is then placed onto a dehydration tray for insertion into a dehydrator.

Utilize dehydration equipment such as the “Harvest Saver R5A” by Commercial Dehydrator Systems, Inc. of Eugene, Oregon, for example. Transfer the food items to the dehydrator trays and spread them out evenly and uniformly. In the Harvest Saver dryers the optimal fill rate is 12-15 lbs per tray, in order to hit an optimal dryer load size of roughly 168-210 lbs per machine, depending on the density of the food item.

Close the dehydrator and dehydrate at 145 degrees F. for 36-54 hours or until the food items hit the target water activity (aW) of 0.25 to 0.35. A water activity meter should be used to measure and assess aW in real time.

Once dehydrated, the food item is allowed to cool to ambient temperature. At this point, the food item should be packaged into an airtight container as quickly as possible to avoid the food item from absorbing atmospheric moisture. The food items can be packaged into an airtight container and sold as a novel shelf stable snack nut/seed/legume. Alternatively, the food items may be ground into a meal, flour, protein powder, or powder for encapsulation as a postbiotic supplement. The meal can also be further ground into a uniform butter and then packaged into a glass jar or other airtight container.

The remaining brine can either be discarded or bottled for sale as a functional probiotic beverage. If the remaining brine is going to be sold as a raw, living product, then steps must be taken in the form of a Critical Control Point (CCP), to ensure the safety and viability of the finished product.

The first step in this process is testing the initial post-fermentation pH, for which the CCP to be met is below 4.0. Once a batch passes pH, the brine must be transferred into buckets/barrels, or tanks and put back into fermentation to be held at 62F for exactly 7 days. The vessels must be filled as much as possible, limiting headspace in order to exclude as much oxygen as possible, and the vessels must also be setup to offgas during this dwell time (ie. buckets/barrels with bungs and airlocks).

Retest the brine for pH. Upon completion of this dwell time, the brine is retested once again, in order to validate that the brine has held a pH of below 4.0 for 7 days. This CCP is based on scientific studies and food safety guidelines that dictate that a raw living product should be either below 3.8 at the end of fermentation, OR that it achieves an initial post fermentation pH of below 4.0, and that it holds below that threshold of 4.0 for 7 days. This ensures that the product is safe for consumption and that any potential pathogens that may have been present at the start of fermentation are eliminated.

Once the brine meets this CCP, immediately move the sealed vessels into refrigeration, where it must be stored and the cold-chain must be maintained throughout the life cycle of the product. The fermentation lids should be replaced with regular, non-vented lids for storage.

The present invention produces novel postbiotic foods that have increased nutritional value, increased bioavailability and digestibility of nutrients, and increased shelf life for the durable and functional nutrition generated by these processes. Simultaneously, it achieves decreased anti-nutrient profiles, decreased agricultural pollutant profiles, decreased toxin profiles, and decreased allergens.

We have found that our novel process inactivates tree nut allergens in almonds and cashews to non-detectable limits (see Columbia lab results below). We also found that our process reduces peanut allergen levels to only 3 ppm, when the normal reporting threshold is 5 ppm (see Biogen lab results below).

The present invention introduces a novel method of sterilizing nuts, seeds, and legumes via a dual kill step that results in up to an 8-log reduction in pathogens of concern without denaturing them, making it a preferable alternative to pasteurization.

Not only does our process tackle the above needs without denaturing the base ingredients or their nutritional compounds, but it also offers added functional benefits. We ran metabolomics analysis using at Utah State University and found that additional benefits include increased nutritional value, including higher B vitamins, phenols, flavonoids, phytochemicals, quercetins, and catechins, decreased sugars, and decreased anti-nutrient profile (including phytic acid and oxalic acid) (see Utah State University lab results below). All of these benefits can be considered postbiotic in nature, as they result specifically from the metabolic actions of the lactic acid bacteria during fermentation.

Our method provides a longer shelf life for almonds, thanks to the lactic acid and the bacteriocins generated by lactic acid bacteria fermentation, and lower moisture content than when they come off the tree thanks to the dehydration.

Based on research on other fermented foods like kimchi, we hypothesize that there is also a decreased agricultural pollutant profile (including glyphosate and other chemically similar agricultural inputs, which can be toxic and carcinogenic).

Based on research on fermentation in almonds, we hypothesize that there is a decrease in toxin profiles (including mycotoxins such as aflatoxins B1 and G1—Appendix D—https://www.sciencedirect.com/science/article/abs/pii/S0023643820304898)

Based on research of postbiotics generated by lactic acid fermentation, we hypothesize that several postbiotic compounds develop and are present in our fermented and dehydrated food products as a result of fermentation including lactic acid, bacteriocins, butyric acid and other short-chain fatty acids, and bioactive peptides. (Appendix E—https://www.ncbi.nlm.nih.gov/pmc/articles/PMC8387231).

We are currently validating the above hypotheses via testing of various nuts, seeds, and legumes produced with our process.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

These and other features will be more readily understood from the following detailed description taken in conjunction with the accompanying drawings, in which:

FIG. 1 is a diagram of a method of the invention;

FIG. 2 is a flow chart of one embodiment of the method of the invention; and

FIG. 3 is a flow chart of another embodiment of the method of the invention.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list. When the word “each” is used to refer to an element that was previously introduced as being at least one in number, the word “each” does not necessarily imply a plurality of the elements, but can also mean a singular element.

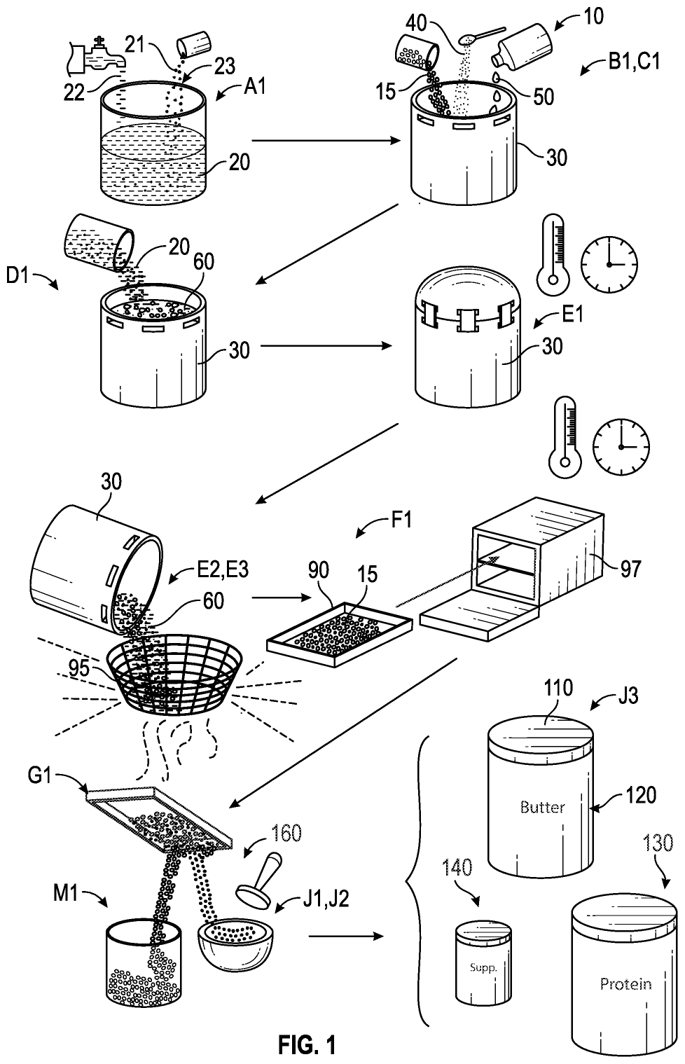

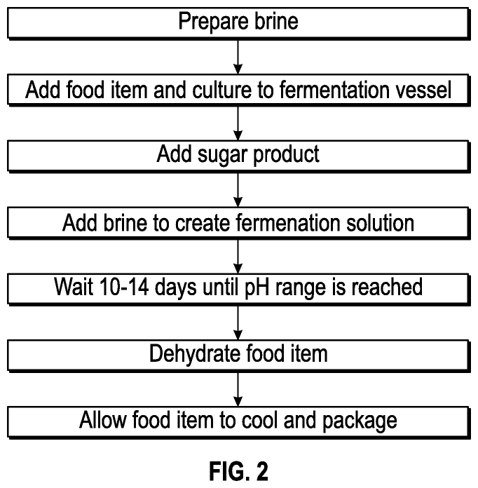

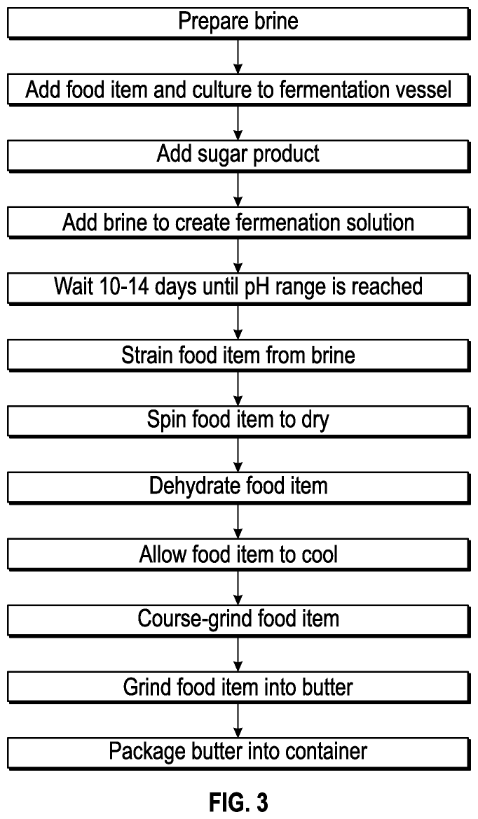

FIGS. 1-3 illustrate an exemplary method 10 of fermenting and dehydrating a food item 15, such as nuts, seeds, or legumes. In a preferred embodiment of the invention, the food item 15 is almonds. The method may utilize cultures of lactic acid bacteria, including strains such as Lactiplantibacillus plantarum, Leuconostoc mesenteroides, and Pediococcus acidilactici. The cultures are a blend of homofermentative and heterofermentative lactic acid bacteria.

Once all equipment used in the method is cleaned and sanitized, in a first step A1 of the invention, a brine solution 20 is prepared that comprises a salt product 21 and water 22. Such a brine solution 20 is preferably prepared in a brine barrel/tank 23 either by hand mixing by using a plastic mixing spoon (not shown), or by using an electric paddle mixer (not shown) through a barrel lid (not shown). If making multiple batches of fermented almonds, for example, as would normally be the case during a production run, a large batch of brine solution 20 may be mixed to accommodate all of the barrels of fermented almonds 15 instead of mixing each batch of brine solution 20 one at a time.

Preferably the salt product 21 is a natural, unrefined salt product containing minerals such as marketed under the brand Redmond Real Salt. The water 22 is preferably purified spring water 22. Redmond real salt 21 contains visible trace minerals which will not go fully into solution regardless of the mixing duration. The salt 21, however, should be fully dissolved, with no salt particles visible to the naked eye. A salometer (not shown) or other comparable instrument can be used to test the salinity of the brine solution 20 in order to verify that the solution hits the target brine strength of exactly 3.0%. If needed, add more salt or water, and retest the salinity until a 3% brine solution is achieved.

Separately, and perhaps concurrently to mixing of the brine solution 20, the food item 15 is added in a step B1 to a fermentation vessel 30, along with a culture of lactic acid bacteria 40, which is preferably a mixed culture of lactic acid bacteria 40 in a concentration of exactly 0.08% of the total recipe by weight. The Leuconostoc strain of lactic acid bacteria 40 initiates fermentation and quickly drops the acidity of the solution, so that scientific criteria for sterilization, namely a finished pH of under 3.8, or a finished pH of 4.0 or below that is held at that level for at least 7 days, is achieved. The heterofermentative lactic acid bacteria 40 is most active in the first 4-5 days of fermentation, which is also important for building a desirable flavor.

Next, in a step C1, a sugar product 50 is added to the fermentation vessel 30. Such a sugar product 50 is preferably date syrup at a concentration of exactly 1.0%. The date syrup 50 has a relatively high viscosity, so in order to get all of the specified quantity of the date syrup 50 into each fermentation vessel 30, the brine solution 20 can be used to dissolve the date syrup 50, lowering its viscosity and making it easier to transfer the total weighed quantity. Conversely, the date syrup 50 can be added to the brine solution 20 prior to mixing in order to lower the viscosity of the syrup and make it easier to transfer and to obtain the desired measurement quantity in each fermentation vessel 30.

After verifying that the salt product 21 is fully dissolved in the brine solution 20, the brine solution 20 is added to the fermentation vessel 30 in a step D1 either manually or with a brine pump (not shown), creating a fermentation solution 60. The brine barrel 23 should be constantly agitated or mixed during this process in order to equally distribute the minerals between the multiple fermentation vessels 30, if more than one fermentation vessel 30 is being prepared. If the brine solution 20 is not agitated properly then some fermentation vessels 30 will likely contain more minerals than others, which will have an effect on the fermentation process, and ultimately lead to inconsistency in the quality and sensory profiles of the finished product from batch to batch. The fermentation solution 60 is then manually mixed within each fermentation vessel 30, preferably with a long plastic spoon (not shown), to equally distribute the ingredients, and particularly the culture of lactic acid bacteria 40, throughout the fermentation solution 60.

If the food item 15 is of the type that floats on the brine solution 20, a plastic strainer or other retainer can be used to hold down the food item 15 within the fermentation vessel 30. In a step E1, the fermentation vessel 30 is sealed and set for between ten to fourteen days in an environment within a first fermentation temperature range, preferably between 60-degrees F. and 64-degrees F., and preferably at a temperature of substantially 62-degrees F.

After ten days the pH of the fermentation solution 60 is checked to see if it is within the acceptable pH range of between 3.3 pH and 4.0 pH. If not, the fermentation solution 60 is allowed to ferment for another two days, after which the pH of the fermentation solution 60 is checked again. If not, the fermentation solution 60 is allowed to ferment for another two days, after which the pH of the fermentation solution 60 is checked again. If the fermentation solution 60 has not reached a pH of 3.8 or below by day 14, it is likely that something has gone wrong, the fermentation solution 60 will likely not reach the desired acceptable pH range, and it should be discarded as waste. Similarly, the fermentation vessel 30 is preferably inspected for any quality defects such as surface yeast or mold, and any batch that shows signs of either should be discarded as waste.

Once the fermentation solution 60 has achieved a pH within the acceptable pH range, in a step E2, the food item 15 is strained in a strainer 95, or the like, to remove the brine solution 20 and other ingredients. The food item 15 may also be spun, such as in a common salad spinner (not shown), to centrifugally remove additional brine solution 20 and other liquids and ingredients. Optionally the fermentation solution 60 without the food item 15 may be refrigerated for future alternate uses.

The food item 15 is then placed onto a dehydration sheet 90 in a step F1, for insertion into a dehydrator 97. Preferably the food item 15 is dehydrated at 145 degrees F. for 36-56 hours or until the food items hit the target aW of 0.25 to 0.35. A water activity meter should be used to measure aW in real time.

Once dehydrated, the food item 15 is allowed to cool to ambient temperature in a step G1. At this point the food item 15 should be packaged into an airtight container 110 as quickly as possible to avoid the food item 15 from absorbing atmospheric moisture. The food item 15 can be packaged into an airtight container and sold as a novel shelf stable snack nut/seed/legume 160. Alternatively, the food item 15 may be ground into a meal, flour, protein powder 150, or powder for encapsulation as a postbiotic supplement 140 in a step J1. The meal can also be further ground into a uniform butter 120 in a step J2 and then packaged into a glass jar or other airtight container in a step J3.

While a particular form of the invention has been illustrated and described, it will be apparent that various modifications can be made without departing from the spirit and scope of the invention. For example, the order of each step illustrated may be altered, or additional steps may be introduced for large batches. Accordingly, it is not intended that the invention be limited, except as by the appended claims.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

### Footnotes and Attached Laboratory/University Study Results in Appendix

### Finished Product Specifications: Fermented Almonds

Expected fermentation dwell time: 10-14 days
Acceptable pH range post fermentation: 3.3-4.0
Acceptable aW range post dehydration: 0.25 to 0.35

### Sensory Metrics:

Flavor: Nutty, lactic sour, salty, basic sour, umami, with lactic sour/fermented notes on finish
Aroma: Grassy green notes, nutty, acetic and lactic acid, briny.
Texture: High level of fracturability. The almond shatters in your mouth, and then comes back together with a medium degree of tooth-stick.
Appearance: Plain whole almonds. Somewhere between a raw and roasted almond in color. No seasoning or coatings.

| | Description | | | | |
|---|---|---|---|---|---|
| Recipe Code | Almond | Date Start | Date finished | | Time/Temp |
| | MultiTank | | | | |
| | Recipe using | | | | |
| | working | | | | |
| | volume of | | | | |
| | 385 liters fill | | | | |
| .05.07.04-A | rate = 99.5% | | | | |
| TOTAL | | Waste % | | | |
| grams | Recipe % | Estimated | IN Grams | In Kilograms | IN Lbs |
| 383075 | | | Net per barrel | Net per barrel | Net per barrel |
| California | 40.00% | 0.0% | 153230.00 | 153.23 | 337.81 |
| Almonds | | | | | |
| (whole) | | | | | |
| Date Syrup | 1.00% | 0.0% | 3830.75 | 3.83 | 8.45 |
| Brine (3.0%) | 59.00% | 0.0% | 226014.25 | 226.01 | 498.28 |
| culture | 0.08% | 0.0% | 306.46 | 0.31 | 0.68 |
| totals | 100.00% | | 383381.5 | 383.38 | 845.2 |
| Recipe Code | Description | Date Start | Date finished | | Time/Temp |
| | Cashew | | | | |
| | MultiTank | | | | |
| | Recipe using | | | | |
| | working | | | | |
| | volume of | | | | |
| | 385 liters fill | | | | |
| .10.06-A | rate = 99.5% | | | | |
| TOTAL | | Waste % | | | |
| grams | Recipe % | Estimated | IN Grams | In Kilograms | IN Lbs |
| 383075 | | | Net per barrel | Net per barrel | Net per barrel |
| Cashews | 44.00% | 0.0% | 168553.00 | 168.55 | 371.60 |
| Date Syrup | 2.25% | 0.0% | 8619.19 | 8.62 | 19.00 |
| Brine (3.0%) | 53.67% | 0.0% | 205596.35 | 205.60 | 453.26 |
| culture | 0.08% | 0.0% | 306.46 | 0.31 | 0.68 |
| totals | 100.00% | | 383075.0 | 383.08 | 844.5 |
| TOTAL | | TOTAL | | TOTAL | |
| grams | Recipe % | grams | Recipe % | grams | Recipe % |
| 1875 | | 1875 | | 1875 | |
| Pumpkin | 44.00% | Sunflower | 44.00% | Cashews | 44.00% |
| Seeds | | Seeds | | | |
| Date | 1.00% | Date | 1.00% | Date | 1.00% |
| Syrup | | Syrup | | Syrup | |
| Brine | 54.92% | Brine | 54.92% | Brine | 54.92% |
| (3.0%) | | (3.0%) | | (3.0%) | |
| culture | 0.08% | culture | 0.08% | culture | 0.08% |
| totals | 100.00% | totals | 100.00% | totals | 100.00% |
| TOTAL | | TOTAL | | TOTAL | |
| grams | Recipe % | grams | Recipe % | grams | Recipe % |
| 1875 | | 1875 | | 1875 | |
| Peanuts | 44.00% | WALNUTS | 44.00% | Macadamia | 44.00% |
| | | | | Nuts | |
| Date | 1.00% | Date | 1.00% | Date | 1.00% |
| Syrup | | Syrup | | Syrup | |
| Brine | 54.92% | Brine | 54.92% | Brine | 54.92% |
| (3.0%) | | (3.0%) | | (3.0%) | |
| culture | 0.08% | culture | 0.08% | culture | 0.08% |
| totals | 100.00% | totals | 100.00% | totals | 100.00% |
| TOTAL | | TOTAL | | TOTAL | |
| grams | Recipe % | grams | Recipe % | grams | Recipe % |
| 1875 | | 1875 | | 1875 | |
| PECANS | 44.00% | BRAZIL | 44.00% | Sunflower | 44.00% |
| | | NUTS | | Seeds | |
| Date | 1.00% | Date | 1.00% | Date | 1.00% |
| Syrup | | Syrup | | Syrup | |
| Brine | 54.92% | Brine | 54.92% | Brine | 54.92% |
| (3.0%) | | (3.0%) | | (3.25%) | |
| culture | 0.08% | culture | 0.08% | culture | 0.08% |
| totals | 100.00% | totals | 100.00% | totals | 100.00% |

These items listed below are the alternate experiments we conducted at the onset of our product development. All of the following experiments were undertaken to determine the best methods and ingredients for lowering the pH, and after extensive experimentation we discovered that date syrup was in fact the most viable means to accomplish a finished pH of 4.0 or below after 10 days of fermentation at 62 degrees Fahrenheit.

Other Experiments Tested

| Recipe Code | Description |
|---|---|
| .01.XX | Whole almonds with culture + |
| | inulin |
| .02.XX | Cracked almonds with |
| | culture |
| .03.XX | Whole almonds with culture + |
| | shredded coconut |
| .04.XX | Whole almonds with culture + |
| | dried seaweed and dried |
| | mushrooms |
| .05.XX | Whole almonds with culture + |
| | medjool date syrup |

Additional studies were conducted and are submitted herewith as appendices to the application, and are included as part of the application herein by reference.

What is claimed is:

1. A method of fermenting and dehydrating a food item, comprising the steps:
adding, in a fermentation vessel, a food item and cultures of lactic acid bacteria;
adding a sugar product to the fermentation vessel;
adding a brine solution comprising a salt product and water to the fermentation vessel to create a fermentation solution;
sealing the fermentation vessel and allowing fermentation within the fermentation vessel within a fermentorium holding a specific temperature range for a specific timeframe, until a pH of the fermentation solution is within a specified acidity range;
straining the nut product from the brine solution;
moving the food item onto a dehydration tray and dehydrating the food item in a dehydrator for a specified timeframe.

2. The method of claim 1, further comprising that the food item is a nut, seed, or legume.

3. The method of claim 1, further comprising that the nut, seed, or legume is an almond, cashew, walnut, brazil nut, macadamia nut, pecan, sunflower seed, pumpkin seed, and peanut.

4. The method of claim 1, wherein the lactic acid bacteria cultures added to the fermentation vessel is at a concentration of substantially 0.08% of the fermentation solution.

5. The method of claim 1, further comprising that the lactic acid bacteria is a blend of homofermentative and heterofermentative strains.

6. The method of claim 1, further comprising that the lactic acid bacteria blend contain strains such as Lactiplantibacillus plantarum, Leuconostoc mesenteroides, and Pediococcus acidilactici.

7. The method of claim 1, further comprising that the sugar product is date syrup added to the fermentation solution at a concentration of substantially 1.0% of the fermentation solution.

8. The method of claim 1, wherein the brine solution comprises a 3% brine solution.

9. The method of claim 1, further comprising that the specified temperature range in the fermentorium is between 60 and 64 degrees Fahrenheit.

10. The method of claim 1, further comprising that the specified timeframe of the fermentation is ten to fourteen days.

11. The method of claim 1, further comprising that the specified target acidity of the fermentation solution after ten to fourteen days is between 3.3 to 4.0 pH.

12. The method of claim 1, further comprising that once target acidity is achieved, operator initiates dehydrating the food item for at least thirty-six to fifty-four hours at 145 degrees Fahrenheit.

13. The method of claim 1, further comprising that the specified level of dryness for the nut/seed/legume is between a water activity of 0.25 aW to 0.35 aW,

14. The method of claim 1, further comprising that the product is removed from the dehydrator for packaging as a novel fermented and dehydrated postbiotic snack nut/seed/legume, comprising the step:
inserting the fermented and dehydrated postbiotic snack nut/seed/legume into a vessel such as a plastic stand up pouch using a machine such as a linear bucket scale.

15. The method of claim 1, further comprising a method of making a fermented and dehydrated nut, seed, or legume butter derived therefrom, comprising the steps:
grinding the nut, seed, or legume into a butter using a machine such as a Cocoatown stone-grinder;
packaging the butter into a vessel such as a plastic or glass container using a depositor machine.

16. The method of claim 1, further comprising a method of making a fermented and dehydrated nut, seed, or legume flour derived therefrom, comprising the steps:
grinding the nut, seed, or legume into a flour using a machine such as a vertical cutter-mixer;
packaging the butter into a vessel such as a plastic stand-up pouch.

17. The method of claim 1, further comprising a method of making a fermented and dehydrated nut, seed, or legume protein powder derived therefrom, comprising the steps:
grinding the nut, seed, or legume into a powder using a machine such as a vertical cutter-mixer;
packaging the butter into a vessel such as a plastic stand-up pouch or plastic cylinder.

18. The method of claim 1, further comprising a method of making a fermented and dehydrated nut, seed, or legume postbiotic encapsulated supplement derived therefrom, comprising the steps:
grinding the nut, seed, or legume into a powder using a machine such as a vertical cutter-mixer;
packaging the butter into a vessel such as a size 1 supplement capsule.

19. The method of claim 1, further comprising a method of making a probiotic fermented nut, seed, or legume brine beverage derived therefrom, comprising the steps:
capturing the strained the nut, seed, or legume brine into a vessel such as a 275 gallon plastic tote;
packaging the brine into a vessel such as a 3 oz plastic shot or a glass jars using a depositor machine.
